(19) United States
(12) Patent Application Publication
(10) Pub. No.: US 2021/0387992 A1
(43) Pub. Date: Dec. 16, 2021

(54) POLYMORPH FORM OF A MONOPHOSPHATE HYDRATE SALT OF A KNOWN TETRAHYDROISOQUINOLINE DERIVATIVE

(71) Applicant: Pfizer Inc., New York, NY (US)

(72) Inventors: Brian Samas, Branford, CT (US); Wesley Clark, Gales Ferry, CT (US)

(73) Assignee: Pfizer Inc., New York, NY (US)

(21) Appl. No.: 17/424,721

(22) PCT Filed: Jan. 20, 2020
(86) PCT No.: PCT/IB2020/050397

§ 371 Date: Jul. 21, 2021

Related U.S. Application Data
(60) Provisional application No. 62/795,631, filed on Jan. 23, 2019.
(60) Provisional application No. 62/872,802, filed on Jul. 11, 2019.

(51) Int. Cl. C07D 487/04; A61K 45/06

(52) U.S. Cl. CPC C07D 487/04; C07B 2200/13; A61K 45/06

(57) ABSTRACT

This invention relates to crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate, and to compositions and therapeutic uses thereof.

### FIELD OF THE INVENTION

The present invention relates to a novel crystalline form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol or a pharmaceutically acceptable salt thereof. More particularly, the present invention relates to novel crystalline form of a pharmaceutically acceptable salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol. The present invention also relates to formulations and therapeutic uses of such a polymorph.

### BACKGROUND

The compound (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol is represented by the formula (I) below.

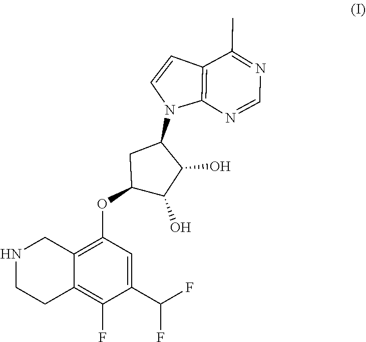 [SMILES: Cc1ncnc2c1ccn2[C@@H]1C[C@H](Oc2cc(C(F)F)c(F)c3c2CNCC3)[C@@H](O)[C@H]1O]

The preparation of the compound (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol as a hydrochloride salt is described in Example 190 of WO2017/212385 and is depicted as follows.

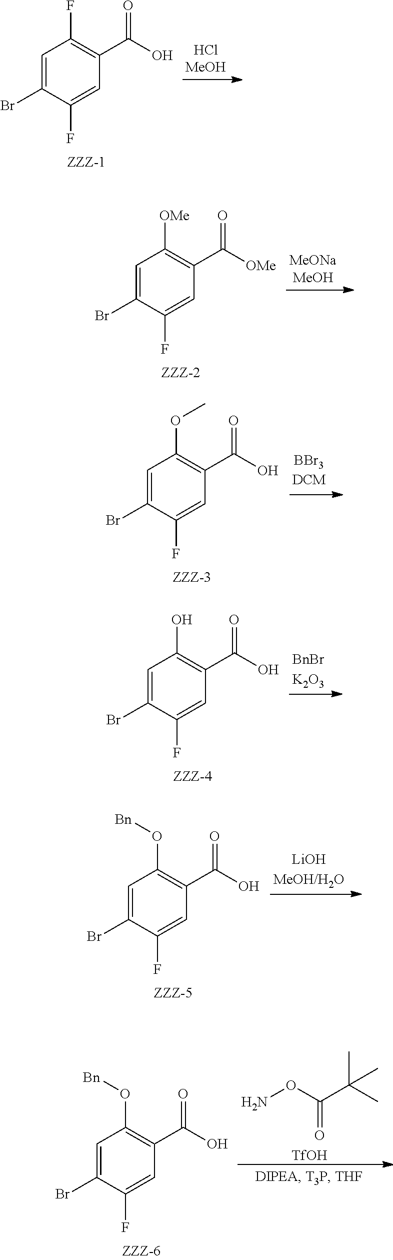

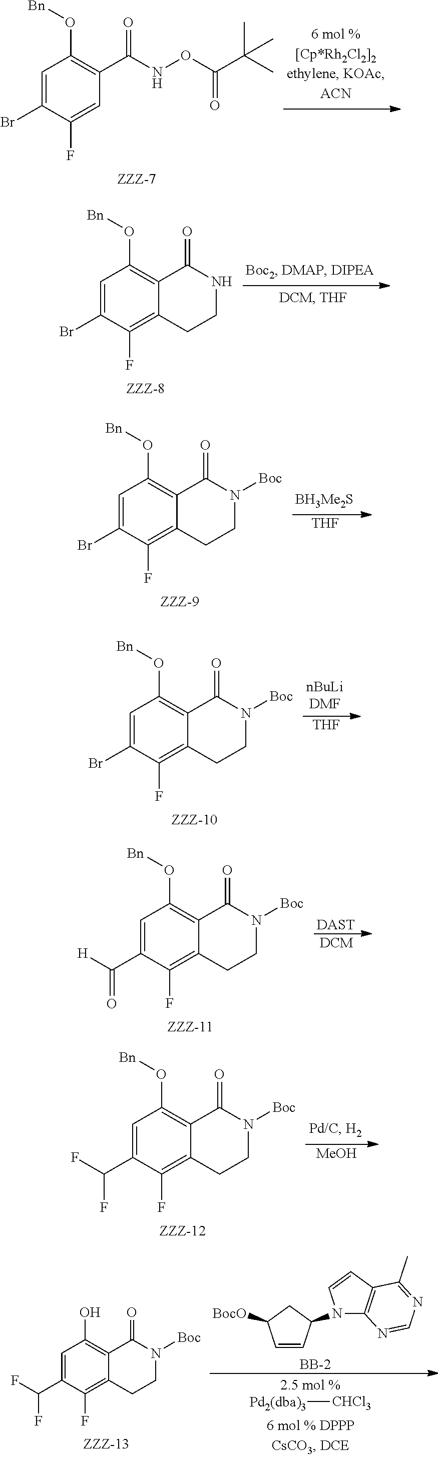

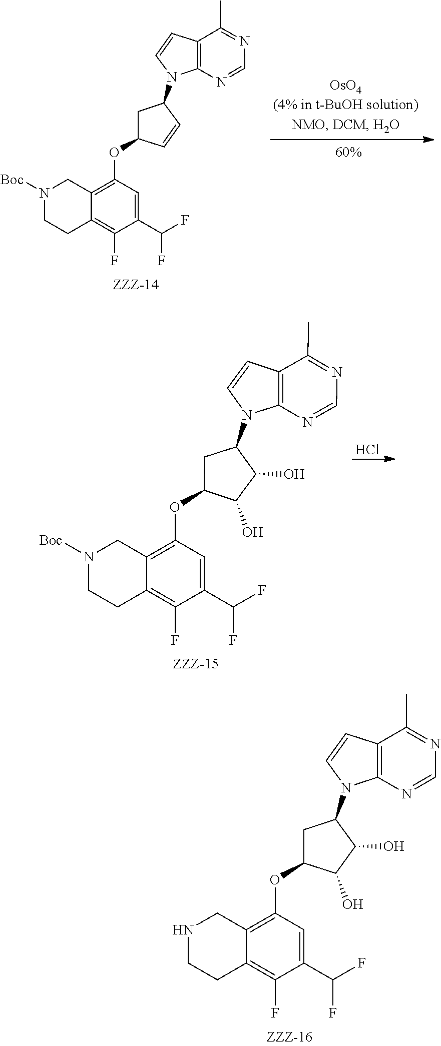 [SMILES: Cc1ncnc2c1ccn2[C@H]1C=C[C@@H](Oc2cc(C(F)F)c(F)c3c2CN(C(=O)OC(C)(C)C)CC3)C1 ; Cc1ncnc2c1ccn2[C@@H]1C[C@H](Oc2cc(C(F)F)c(F)c3c2CN(C(=O)OC(C)(C)C)CC3)[C@@H](O)[C@H]1O ; Cl ; Cc1ncnc2c1ccn2[C@@H]1C[C@H](Oc2cc(C(F)F)c(F)c3c2CNCC3)[C@@H](O)[C@H]1O]

In this procedure tert-butyl 6-(difluoromethyl)-8-(((1S,2S,3S,4R)-2,3-dihydroxy-4-(4-methyl-7H-pyrrolo[2,3-d]pyrimidin-7-yl)cyclopentyl)oxy)-5-fluoro-3,4-dihydroisoquinoline-2(1H)-carboxylate is first deprotected in a mixture of dichloromethane and dioxane/HCl. The solid precipitated is separated, dried and lyophilised to provide a hydrochloride salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol as “a light yellow solid”. The specific solid form of the hydrochloride salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol prepared is not specified. (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol may be prepared from this salt by standard basification techniques.

The preparation of the compound (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol as a hydrochloride salt as described in Example 190 of WO2017/212385 was replicated as closely as possible in Reference Example 1 herein. PXRD and elemental analysis shows the product obtained in Example 190 to be an amorphous, dihydrochloride having approximately 1 mol water per mol of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol.

In WO2017/212385 (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol is described as a PRMT5 inhibitor useful in the treatment of abnormal cell growth in mammals, especially humans, particularly for the treatment of cancer.

Human cancers comprise a diverse array of diseases that collectively are one of the leading causes of death in developed countries throughout the world (American Cancer Society, Cancer Facts and Figures 2005. Atlanta: American Cancer Society; 2005). The progression of cancers is caused by a complex series of multiple genetic and molecular events including gene mutations, chromosomal translocations, and karyotypic abnormalities (Hanahan & Weinberg, The hallmarks of cancer. Cell 2000; 100: 57-70). Although the underlying genetic causes of cancer are both diverse and complex, each cancer type has been observed to exhibit common traits and acquired capabilities that facilitate its progression. These acquired capabilities include dysregulated cell growth, sustained ability to recruit blood vessels (i.e., angiogenesis) and the ability of tumor cells to spread locally as well as metastasize to secondary organ sites (Hanahan & Weinberg 2000 above). Therefore, the ability to identify novel therapeutic agents that inhibit molecular targets that are altered during cancer progression, or target multiple processes that are common to cancer progression in a variety of tumors, presents a significant unmet need.

Post-translational modification of arginine residues by methylation is important for many critical cellular processes including chromatin remodelling, gene transcription, protein translation, signal transduction, RNA splicing and cell proliferation. Arginine methylation is catalysed by protein arginine methyltransferase (PRMT) enzymes. There are nine PRMT members in all, and eight have reported enzymatic activity on target substrates.

The protein arginine methyltransferase (PRMT) family of enzymes utilize S-adenosyl methionine (SAM) to transfer methyl groups to arginine residues on target proteins. Type I PRMTs catalyse the formation of mono-methyl arginine and asymmetric di-methyl arginines, while Type II PRMTs catalyze mono-methyl arginine and symmetric di-methyl arginines. PRMT5 is a Type II enzyme, twice transferring a methyl group from SAM to the two ω-guanidino nitrogen atoms of arginine, leading to co-NG, N′G di-symmetric methylation of protein substrates.

PRMT5 protein is found in both the nucleus and cytoplasm, and has multiple protein substrates such as histones, transcription factors and spliceosome proteins. PRMT5 has a binding partner, Mep50 (methylosome protein 50) and functions in multiple protein complexes. PRMT5 is associated with chromatin remodeling complexes (SWI/SNF, NuRD) and epigenetically controls genes involved in development, cell proliferation, and differentiation, including tumor suppressors, through methylation of histones (Karkhanis, V. et al., Versatility of PRMT5 Induced Methylation in Growth Control and Development, Trends Biochem Sci 36(12) 633-641 (2011)). PRMT5 also controls gene expression through association with protein complexes that recruit PRMT5 to methylate several transcription factors—p53 (Jansson, M. et al., Arginine Methylation Regulates the p53 Response, Nat. Cell Biol. 10, 1431-1439 (2008)); E2F1 (Zheng, S. et al., Arginine Methylation-Dependent Reader-Writer Interplay Governs Growth Control by E2F-1, Mol Cell 52(1), 37-51 (2013)); HOXA9 (Bandyopadhyay, S. et al., HOXA9 Methylation by PRMT5 is Essential for Endothelial Cell Expression of Leukocyte Adhesion Molecules, Mol. Cell. Biol. 32(7):1202-1213 (2012)); and NFκB (Wei, H. et al., PRMT5 dimethylates R30 of the p65 Subunit to Activate NFκB, PNAS 110(33), 13516-13521 (2013)). In the cytoplasm, PRMT5 has a diverse set of substrates involved in other cellular functions including RNA splicing (Sm proteins), golgi assembly (gm130), ribosome biogenesis (RPS10), piRNA mediated gene silencing (Piwi proteins) and EGFR signaling (Karkhanis, 2011).

Additional papers relating to PRMT5 include: Aggarwal, P. et al., (2010) Nuclear Cyclin D1/CDK4 Kinase Regulates CUL4B Expression and Triggers Neoplastic Growth via Activation of the PRMT5 Methyltransferase, Cancer Cell 18: 329-340; Bao, X. et al., Overexpression of PRMT5 Promotes Tumor Cell Growth and is Associated with Poor Disease Prognosis in Epithelial Ovarian Cancer, J Histochem Cytochem 61: 206-217 (2013); Cho E. et al., Arginine Methylation Controls Growth Regulation by E2F1, EMBO J. 31(7) 1785-1797 (2012); Gu, Z. et al., Protein Arginine Methyltransferase 5 Functions in Opposite Ways in the Cytoplasm and Nucleus of Prostate Cancer Cells, PLoS One 7(8) e44033 (2012); Gu, Z. et al., Protein Arginine Methyltransferase 5 is Essential for Growth of Lung Cancer Cells, Biochem J. 446: 235-241 (2012); Kim, J. et al., Identification of Gastric Cancer Related Genes Using a cDNA Microarray Containing Novel Expressed Sequence Tags Expressed in Gastric Cancer Cells, Clin Cancer Res. 11(2) 473-482 (2005); Nicholas, C. et al., PRMT5 is Upregulated in Malignant and Metastatic Melanoma and Regulates Expression of MITF and p27(Kip1), PLoS One 8(9) e74710 (2012); Powers, M. et al., Protein Arginine Methyltransferase 5 Accelerates Tumor Growth by Arginine Methylation of the Tumor Suppressor Programmed Cell Death 4, Cancer Res. 71(16) 5579-5587 (2011); Wang, L. et al., Protein Arginine Methyltransferase 5 Suppresses the Transcription of the RB Family of Tumor Suppressors in Leukemia and Lymphoma Cells, Mol. Cell Biol. 28(20), 6262-6277 (2008).

PRMT5 is overexpressed in many cancers and has been observed in patient samples and cell lines including B-cell lymphoma and leukemia (Wang, 2008) and the following solid tumors: gastric (Kim 2005) esophageal (Aggarwal, 2010), breast (Powers, 2011), lung (Gu, 2012), prostate (Gu, 2012), melanoma (Nicholas 2012), colon (Cho, 2012) and ovarian (Bao, 2013). In many of these cancers, overexpression of PRMT5 correlated with poor prognosis. Aberrant arginine methylation of PRMT5 substrates has been linked to other indications in addition to cancer, such as metabolic disorders, inflammatory and autoimmune disease and hemaglobinopathies.

Given its role in regulating various biological processes, PRMT5 is an attractive target for modulation with small molecule inhibitors such as (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol.

### SUMMARY

Polymorphs are different crystalline forms of the same compound. The term polymorph may or may not include other crystalline solid state molecular forms including hydrates (e.g., bound water present in the crystalline structure) and solvates (e.g., bound solvents other than water present in the crystalline structure) of the same compound. Polymorphs typically have different crystal structures due to a different packing of the molecules in the lattice. This results in a different crystal symmetry and/or unit cell parameters which directly influences its physical properties such as the X-ray diffraction characteristics of crystals or powders.

Polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since any impurities present may produce undesired toxicological effects. Certain polymorphic forms may also exhibit enhanced (e.g. thermodynamic) stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

For pharmaceutical development and commercialisation, there is a need to identify a solid form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol, or a pharmaceutically acceptable salt or solvate thereof, that can be readily manufactured, processed and formulated. Consequently, there is a need to identify a solid form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol, or a pharmaceutically acceptable salt or solvate thereof, having desirable physicochemical and manufacturing properties.

The present invention provides a novel crystalline form of a pharmaceutically acceptable salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol. More particularly, the present invention relates to crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate having desirable properties such as high crystallinity, high purity, and favorable physical stability, chemical stability, dissolution and mechanical properties. In particular, crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate provides improved physical stability (including low hygroscopicity) relative to the hydrochloride salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol disclosed in WO2017/212385.

### BRIEF DESCRIPTION OF THE DRAWINGS

FIG. 1 shows the PXRD pattern of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate.

FIG. 2 shows the ¹³C solid state NMR spectrum of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

FIG. 3 shows the ¹⁹F solid state NMR spectrum of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

FIG. 4 shows the FT Raman spectrum of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

FIG. 5 shows the structure of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate (protons on OW3 in FIG. 5 are not depicted) as determined by using single crystal X-ray diffraction

FIG. 6 shows the PXRD pattern of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate synthesized in Example 1A.

FIG. 7 shows the PXRD pattern of amorphous (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol (di) hydrochloride synthesized in Reference Example 1, and further discussed in Reference Examples 2 and 3.

FIG. 8 shows PXRD patterns of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol (di) hydrochloride synthesized in Reference Example 1 prior to exposure to 80% RH (amorphous, single defined peak is a likely an artifact of sample preparation) (A); and after storage at greater than 80% RH (crystalline) (B).

### DETAILED DESCRIPTION

The present invention may be understood more readily by reference to the following detailed description of the embodiments of the invention and the Examples and Figures included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta).

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K alpha (wavelength 1.54 Å) radiation comprising characterising peaks at about 5.8, 10.5 and 10.7 degrees 2-theta (+/−0.2 degrees 2-theta).

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 11.5 and 17.5 degrees 2-theta (+/−0.2 degrees 2-theta).

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 10.5, 10.7 and 17.5 degrees 2-theta (+/−0.2 degrees 2-theta).

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 10.5, 10.7, 11.5 and 17.5 degrees 2-theta (+/−0.2 degrees 2-theta).

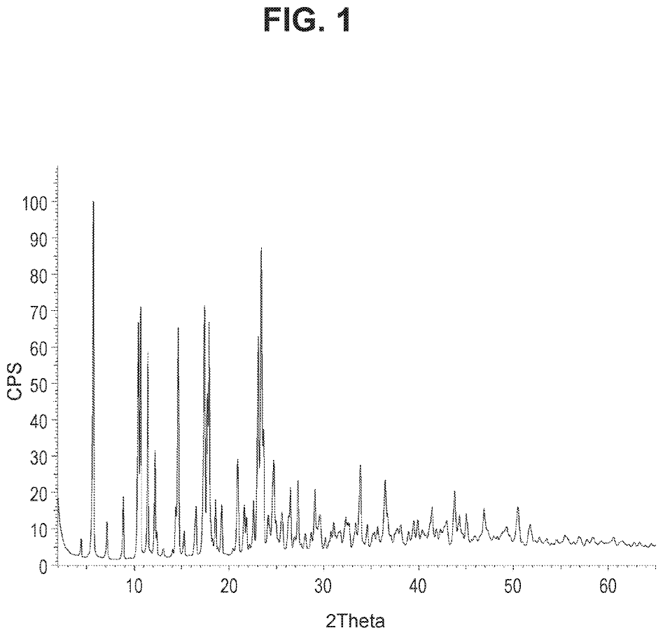

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 8.9, 10.5, 10.7, 11.5 and 17.5 degrees 2-theta (+/−0.2 degrees 2-theta). In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation essentially the same as shown in FIG. 1.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation having a PXRD peak listing essentially the same as in Table 1.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 40.1, 123.5 and 149.3 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 40.1, 121.3, 123.5 and 149.3 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 40.1, 121.3, 123.5, 149.3 and 151.3 ppm±0.2 ppm.

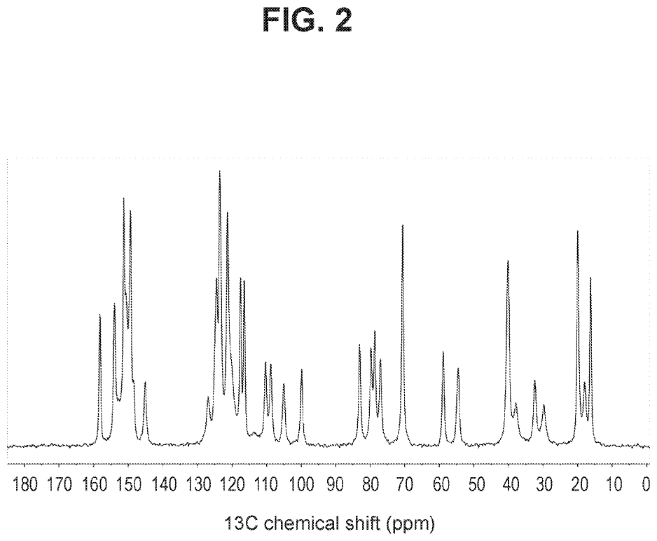

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum essentially the same as shown in FIG. 2.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum peak listing essentially the same as in Table 2.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum comprising a characterising peak at about −129.6 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum comprising characterising peaks at about −129.6 and −128.4 ppm±0.2 ppm.

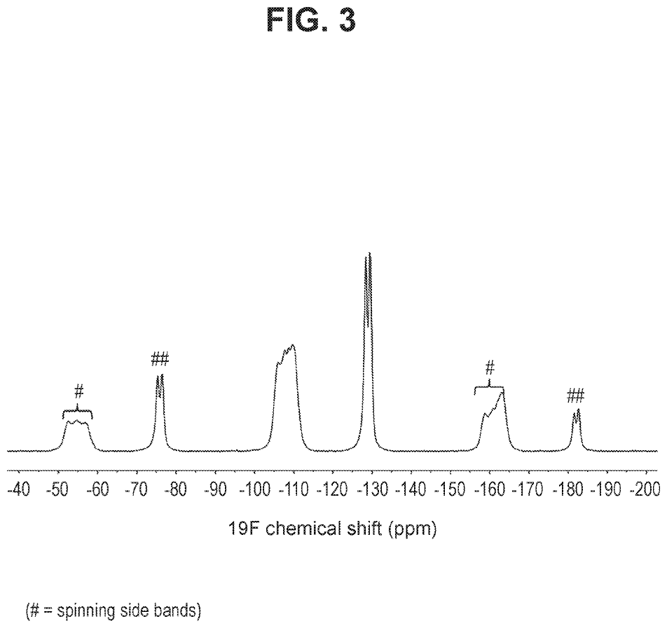

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum essentially the same as shown in FIG. 3.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum peak listing essentially the same as in Table 3.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum comprising characterising peaks at about 702, 1604 and 1630 cm⁻¹±2 cm⁻¹.

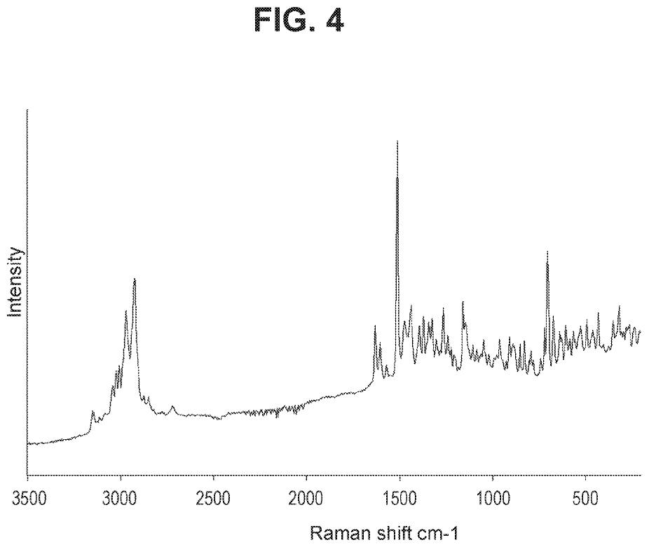

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum essentially the same as shown in FIG. 4.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum peak listing essentially the same as in Table 4.

Each of the embodiments of the present invention described above can be combined with any other embodiment of the present invention described herein not inconsistent with the embodiment with which it is combined. Examples of such combinations are provided below.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta) and by a ¹³C-ssNMR spectrum comprising characterising peaks at about 123.5 and 149.3 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta) and by a ¹⁹F-ssNMR spectrum comprising a characterising peak at about −129.6 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta) and by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm and by a ¹⁹F-ssNMR spectrum comprising a characterising peak at about −129.6 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm and by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum comprising a characterising peak at about −129.6 ppm±0.2 ppm and by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹³C-ssNMR spectrum comprising characterising peaks at about 123.5 and 149.3 ppm±0.2 ppm and by a ¹⁹F-ssNMR spectrum comprising a characterising peak at about −129.6 ppm±0.2 ppm.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹³C-ssNMR spectrum comprising characterising peaks at about 123.5 and 149.3 ppm±0.2 ppm and by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹⁹F-ssNMR spectrum comprising a characterising peak at about −129.6 ppm±0.2 ppm and by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm, by a ¹⁹F-ssNMR spectrum comprising a characterising peak at about −129.6 ppm±0.2 ppm and by a FT Raman spectrum comprising characterising peaks at about 702 and 1630 cm⁻¹±2 cm⁻¹.

In one embodiment, the invention provides crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹³C-ssNMR spectrum comprising characterising peaks at about 123.5 and 149.3 ppm±0.2 ppm, by a ¹⁹F-ssNMR spectrum comprising a characterising peak at about −129.6 ppm±0.2 ppm and by a FT Raman spectrum comprising characterising peaks at about 702 and 1630 cm⁻¹+2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta).

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 10.5 and 10.7 degrees 2-theta (+/−0.2 degrees 2-theta).

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 11.5 and 17.5 degrees 2-theta (+/−0.2 degrees 2-theta).

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 10.5, 10.7 and 17.5 degrees 2-theta (+/−0.2 degrees 2-theta).

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 10.5, 10.7, 11.5 and 17.5 degrees 2-theta (+/−0.2 degrees 2-theta).

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising characterising peaks at about 5.8, 8.9, 10.5, 10.7, 11.5 and 17.5 degrees 2-theta (+/−0.2 degrees 2-theta).

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation essentially the same as shown in FIG. 1.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation having a PXRD peak listing essentially the same as in Table 1.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 40.1, 123.5 and 149.3 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 40.1, 121.3, 123.5 and 149.3 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 40.1, 121.3, 123.5, 149.3 and 151.3 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum essentially the same as shown in FIG. 2.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum peak listing essentially the same as in Table 2.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum comprising a characterising peak at about −129.6 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum comprising characterising peaks at about −129.6 and −128.4 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum essentially the same as shown in FIG. 3.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum peak listing essentially the same as in Table 3.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum comprising characterising peaks at about 702, 1604 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum essentially the same as shown in FIG. 4.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a FT Raman spectrum peak listing essentially the same as in Table 4.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta) and by a ¹³C-ssNMR spectrum comprising characterising peaks at about 123.5 and 149.3 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta) and by a ¹⁹F-ssNMR spectrum comprising a characterising peak at about −129.6 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta) and by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm and by a ¹⁹F-ssNMR spectrum comprising a characterising peak at about −129.6 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm and by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹⁹F-ssNMR spectrum comprising a characterising peak at about −129.6 ppm±0.2 ppm and by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹³C-ssNMR spectrum comprising characterising peaks at about 123.5 and 149.3 ppm±0.2 ppm and by a ¹⁹F-ssNMR spectrum comprising a characterising peak at about −129.6 ppm±0.2 ppm.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹³C-ssNMR spectrum comprising characterising peaks at about 123.5 and 149.3 ppm±0.2 ppm and by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹⁹F-ssNMR spectrum comprising a characterising peak at about −129.6 ppm±0.2 ppm and by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a ¹³C-ssNMR spectrum comprising characterising peaks at about 123.5 and 149.3 ppm±0.2 ppm, by a ¹⁹F-ssNMR spectrum comprising a characterising peak at about −129.6 ppm±0.2 ppm and by a FT Raman spectrum comprising characterising peaks at about 702 and 1630 cm⁻¹±2 cm⁻¹.

In one embodiment, the invention provides substantially pure crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate characterised by a PXRD pattern measured using Cu K-alpha (wavelength 1.54 Å) radiation comprising at least 3 characterising peaks selected from about 5.8, 10.5, 10.7, 11.5 and 17.5 degrees 2-theta (+/−0.2 degrees 2-theta), by a ¹³C-ssNMR spectrum comprising characterising peaks at about 123.5 and 149.3 ppm±0.2 ppm, by a ¹⁹F-ssNMR spectrum comprising a characterising peak at about −129.6 ppm±0.2 ppm and by a FT Raman spectrum comprising characterising peaks at about 702 and 1630 cm⁻¹+2 cm⁻¹.

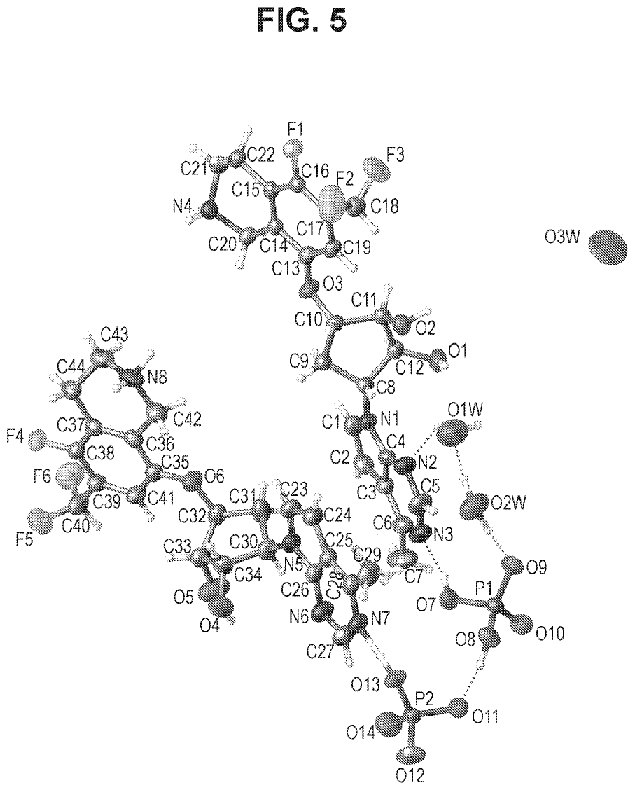

As used herein, the term:
“abnormal cell growth”, unless otherwise indicated, refers to cell growth that is independent of normal regulatory mechanisms (e.g., loss of contact inhibition). Abnormal cell growth may be benign (not cancerous), or malignant (cancerous). In frequent embodiments of the methods provided herein, the abnormal cell growth is cancer.
“cancer” refers to any malignant and/or invasive growth or tumor caused by abnormal cell growth. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from the latter one.
“about” means having a value falling within an accepted standard of error of the mean, when considered by one of ordinary skill in the art.
“crystalline” means having three-dimensional order, i.e. a regularly repeating arrangement of molecules or external face planes. Crystalline forms (polymorphs) may differ with respect to thermodynamic stability, physical parameters, x-ray structure and characteristics, and preparation processes.
“essentially the same” means that variability typical for a particular method is taken into account. For example, with reference to X-ray diffraction peak positions, the term “essentially the same” means that typical variability in peak position and intensity are taken into account. One skilled in the art will appreciate that the peak positions (28) will show some variability, typically ±0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only. Similarly, Raman spectrum wavenumber (cm⁻¹) values show variability, typically as much as ±2 cm⁻¹, while ¹³C and ¹⁹F solid state NMR spectral peaks (ppm) show variability, typically ±0.2 ppm.
“mammal” refers to a human or animal subject. In certain preferred embodiments, the mammal is a human.
“hydrate”, in the context of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate, means having a stoichiometric or non-stoichiometric amount of water bound in the crystal lattice by non-covalent intermolecular bonds. The hydrate state that has been observed to exist for this polymorph includes stoichiometry in the range of about 1.0 to about 1.4 molar equivalents of water per mole of the active moiety between 10% RH to 90% RH at 25° C. By way of example this is illustrated by the molecular structure determination of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate using single crystal X-ray diffraction (see FIG. 5) which indicates that the material analysed is a hydrate of a monophosphate salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol. For the crystal of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate used to generate the structure of FIG. 5, the stoichiometry of the water is about 1.1 moles of water to 1 mole of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate. There is a water molecule (“O3W” in FIG. 5, protons not depicted) with low occupancy in the structure. See also Example 9.
“pharmaceutically acceptable” “carrier”, “diluent”, “vehicle”, or “excipient” refers to a material (or materials) that may be included with a particular active pharmaceutical agent to form a pharmaceutical composition, and may be solid or liquid. Exemplary of solid excipients or carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.
“substantially pure” is to be interpreted as the presence of equal or above 90%, equal or above 95%, equal or above 98%, or equal or above 99% weight/weight of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as compared to any other physical form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol or a pharmaceutically acceptable salt or solvate thereof.
“therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.
“treating”, as used herein, unless otherwise indicated, means reversing, alleviating, inhibiting the progress of, or preventing (i.e. prophylactic treatment) the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. With regard particularly to cancer, these terms simply mean that the life expectancy of an individual affected with a cancer will be increased or that one or more of the symptoms of the disease will be reduced.
term “2-theta value” or “2θ” refers to the peak position in degrees based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2-theta (2θ). It should be understood that reference herein to specific 2θ values for a specific polymorphic form is intended to mean the 2θ values (in degrees) as measured using the X-ray diffraction experimental conditions as described herein. For example, as described herein, Cu K-alpha 1 (wavelength 1.54 Å) was used as the source of radiation.

Also provided by the present invention is a pharmaceutical composition comprising a crystalline form as described herein and a pharmaceutically acceptable carrier or excipient.

Additionally, provided by the present invention are methods of treatment of abnormal cell growth in a mammal comprising administering to the mammal a therapeutically effective amount of a crystalline form as described herein, or composition thereof.

Further provided by the present invention is a crystalline form as described herein, or composition thereof, for use as a medicament or for use in the treatment of abnormal cell growth in a mammal.

Further still, the present invention provides for the use of a crystalline form as described herein, or composition thereof, for the preparation of a medicament useful in the treatment of abnormal cell growth in a mammal.

The abnormal cell growth may be cancer. The cancer referred to herein may be lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma.

The crystalline form as described herein can be administered alone or as a formulation in association with one or more pharmaceutically acceptable carriers or excipients. The choice of excipient will to a large extent depend on factors such as the particular mode of administration, the effect of the excipient on solubility and stability, and the nature of the dosage form.

It will be appreciated that when a crystalline form as described herein is dissolved for formulation purposes, the crystal lattice is no longer present. In this situation the reference to active compound of a crystalline form as described herein below means the (therapeutically active) compound of a crystalline form as described herein.

Pharmaceutical compositions suitable for the delivery of the crystalline form as described herein and their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The crystalline form as described herein may be administered orally. Oral administration may involve swallowing, so that the crystalline form enters the gastrointestinal tract, or buccal or sublingual administration may be employed by which the crystalline form enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The crystalline form as described herein may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the crystalline form as described herein may make up from 0.5 wt (weight) % to 80 wt % of the dosage form, more typically from 0.5 wt % to 20 wt % of the dosage form. In addition to the drug, the tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 2 wt % to 10 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets contain up to about 80 wt % of a crystalline form as described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled targeted- and programmed-release.

Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298.

The crystalline form as described herein may also be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular and subcutaneous. Suitable devices for parenteral administration include needle (including micro-needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of the crystalline form as described herein used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted- and programmed-release. Thus a crystalline form as described herein may be formulated as a solid, semi-solid, or thixotropic liquid for administration as an implanted depot providing modified release of the active compound. Examples of such formulations include drug-coated stents and PGLA microspheres.

The crystalline form as described herein may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro-needle or needle-free (e.g. Powderject™, Bioject™, etc.) injection.

Formulations for topical administration may be formulated to be immediate and/or modified release. Modified release formulations include delayed-, sustained-, pulsed-, controlled targeted- and programmed-release.

The crystalline form as described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The pressurised container, pump, spray, atomiser, or nebuliser contains a solution or suspension of the crystalline form as described herein comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the crystalline form as described herein, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the crystalline form as described herein per actuation and the actuation volume may vary from 1 μL to 100 μL. A typical formulation includes a crystalline form as described herein, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid) (PGLA). Modified release formulations include delayed-, sustained-, pulsed-, controlled-, targeted- and programmed-release.

In the case of dry powder inhalers and aerosols, the dosage unit is determined by means of a valve which delivers a metered amount. Units in accordance with the invention are typically arranged to administer a metered dose or “puff” containing a desired mount of the crystalline form as described herein. The overall daily dose may be administered in a single dose or, more usually, as divided doses throughout the day.

A crystalline form as described herein may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted- and programmed-release.

A crystalline form as described herein may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted-, or programmed-release.

A crystalline form as described herein may be combined with soluble macromolecular entities, such as cyclodextrin and suitable derivatives thereof or polyethylene glycol-containing polymers, in order to improve solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in WO 91/11172, WO 94/02518 and WO 98/55148.

The amount of the active compound of a crystalline form as described herein to be administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day.

Inasmuch as it may desirable to administer a combination of a crystalline form as described herein and a further anti-cancer compound, for example, for the purpose of treating a particular disease or condition, it is within the scope of the present invention that two or more pharmaceutical compositions, at least one of which contains a active compound of a crystalline form as described herein, may conveniently be combined in the form of a kit suitable for co-administration of the compositions. Thus the kit of the invention includes two or more separate pharmaceutical compositions, at least one of which contains a crystalline form as described herein, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The present invention is described with reference to the following Examples. It is to be understood that the scope of the present invention is not limited by the scope of the following Examples.

### Example 1A

### Synthesis of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

A reactor was charged with (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol (6.53 g, 14.56 mmol) followed by 2-propanol (9.3 mL/g, 61 mL). Water (4.14 mL/g, 27.0 mL) was then charged at ambient temperature (ca. 25° C.) and the resulting solution was warmed to 40° C. A solution of phosphoric acid (85% wt/wt in water, 1.1 mol equiv, 16.02 mmol, 1.1 mL) in 2-propanol (3 mL/g, 19.6 mL) was slowly charged over at least 10 minutes. The solution was then warmed to 70° C. and 2-propanol (8.78 mL/g, 57.3 mL) was charged via an addition funnel over at least 10 minutes. At this point, crystallisation self-initiated and the mixture was held at about 65° C. for 2 hours. It was then cooled to about 10° C. over 4 hours, warmed to about 50° C. over 2 hours, held at about 50° C. for 2 hours and finally cooled to 10° C. following a −0.1° C./min ramp. After stirring at about 10° C. for at least 2 hours, the slurry was filtered and the cake was washed with cold 2-propanol/water 95:5 v/v (2 mL/g, 13.1 mL). The cake was then allowed to dry on the filter, under reduced pressure, for at least 1 hour, to afford the title compound as an off-white solid (7.73 g, 13.7 mmol, 94%).

### Example 1B

### PXRD Analysis of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

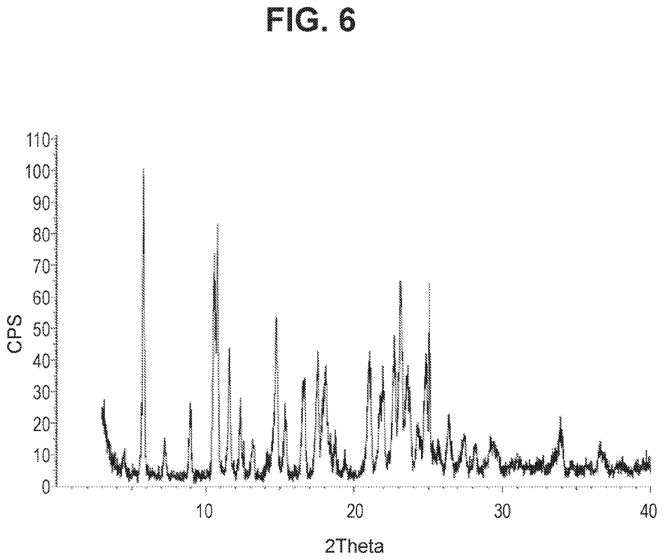

The material was characterized by Powder Diffraction conducted using a Bruker D2 diffractometer equipped with a Cu radiation source, fixed slits (divergence=0.2) and a Lynxeye detector. Data was collected in the Theta-Theta goniometer at the Cu K-alpha (wavelength 1.54 Å) from 3.0 to 40.0 degrees 2-Theta using a Step Size of 0.0141 degrees and a Step Time of 0.5 second. X-ray tube voltage and amperage were set at 30 kV and 10 mA respectively. Samples were prepared by placement in an acrylic sample holder provided by Bruker and rotated during data collection (30 RPM). The PXRD data was read and analyzed in Eva Diffraction software version 4.2.1. The peak search was carried out manually for all intense peak in range 2 to 25 2-theta. The peak selection was carefully checked to ensure that all main peaks had been captured and peak position represents a center point of the peak. Peak shoulders were omitted from the peak selection. The crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate of Example 1A was characterised by PXRD analysis and had a pattern and characterising peak listing essentially in conformity with FIG. 1 and Table 1 (see Example 4), respectively. Specifically, the PXRD pattern for this Example is provided in FIG. 6. The peak list is provided in Table 5.

TABLE 5

| Angle °2-theta | % Relative Intensity |
|---|---|
| 4.5 | 6.2 |
| 5.8 | 100.0 |
| 7.2 | 11.9 |
| 8.9 | 22.5 |
| 10.5 | 66.8 |
| 10.7 | 60.0 |
| 11.5 | 28.6 |
| 12.2 | 15.1 |
| 13.1 | 7.7 |
| 14.7 | 41.4 |
| 15.3 | 15.9 |
| 16.6 | 25.1 |
| 17.5 | 34.2 |
| 18.0 | 31.9 |
| 18.6 | 6.1 |
| 19.3 | 1.7 |
| 21.0 | 31.9 |
| 21.6 | 9.3 |
| 22.7 | 41.8 |
| 23.1 | 58.8 |
| 24.8 | 30.2 |
| 25.1 | 59.3 |

### Example 2

### Pharmaceutical Formulations of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

Prototype formulation blends comprising crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate may be prepared using conventional excipients commonly used in pharmaceutical tablet formulations. Tablets typically contain from 0.5-30% wt/wt of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate. Microcrystalline cellulose and dibasic calcium phosphate anhydrous may be used as tablet fillers, and sodium starch glycolate may be used as a disintegrant. Magnesium stearate may be used as a lubricant.

A typical tablet formulation is provided in Table A.

TABLE A

| Component | Role | wt/wt % |
|---|---|---|
| Crystalline (1S,2S,3S,5R)-3-((6- | API | 3.8 |
| (difluoromethyl)-5-fluoro-1,2,3,4- | | |
| tetrahydroisoquinolin-8-yl)oxy)-5-(4-methyl-7H- | | |
| pyrrolo[2,3-d]pyrimidin-7-yl)cyclopentane-1,2- | | |
| diol monophosphate hydrate | | |
| Cellulose | Filler | 61.4 |
| [Avicel PH 102 (Trade Mark)] | | |
| Dibasic Calcium Phosphate | Filler | 30.8 |
| [DiCAFOS A12 (Trade Mark)] | | |
| Sodium starch glycolate | Disintegrant | 3.0 |
| [Explotab (Trade Mark)] | | |
| Magnesium stearate | Lubricant | 1.0 |

### Example 3

### Synthesis of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

An ambient solution of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol (2.30 Kg, 1.0 equiv., 5.13 mol) in 2-propanol (9.3 L/Kg, 21.3 L) and water (4.14 L/Kg, 9.5 L) was warmed to 40° C. To this warm solution was charged a solution of phosphoric acid (85% w/w) in water, 0.64 Kg, 1.1 equiv.) in 2-propanol (3 L/Kg, 6.9 L) via a head tank over at least 10 minutes. The container of the phosphoric acid solution was rinsed with 2-propanol (0.5 L) and this rinse was charged into the reactor. At this point, the pH of the solution was within the 3.5-4.5 range. The resulting solution was then warmed to 70° C. and 2-propanol (8.78 L/Kg, 20 L) was charged via a head tank over at least 45 minutes. At this point, granulation occurred and the mixture was held at 65° C. for 2 h. It was then cooled to 10° C. over at least 4 h, warmed to 50° C. over at least 2 h, held at 50° C. for at least 2 h and finally cooled to 10° C. following a −0.1° C./min ramp. After stirring at 10° C. for at least 7 h, the slurry was filtered on a Nutsche (Trade Mark) filter, and the cake was washed with cold 2-propanol/water (95:5 v/v, 4.63 L). The cake was then allowed to dry on the filter, under reduced pressure, for at least 1 h. In the meantime, 2-propanol (2.17 L/Kg, 5 L) and water (2.17 L/Kg, 5 L) were successively charged in the reactor and the mixture was heated at 80° C. over at least 30 minutes to help solubilise solids on the walls of the reactor. The solution was held at 80° C. for at least 1 h, then cooled to 20° C. over at least 30 minutes. At this point, 2-propanol (4.35 L/Kg, 10 L) was charged to the reactor via a head tank, followed by the wet cake of solids and 2-propanol (2.17 L/Kg, 5 L) to rinse off the walls. The mixture was heated at 50° C. for at least 30 minutes, held at 50° C. for at least 1 h, cooled to 10° C. over at least 2 h, heated to 50 over at least 30 minutes, held at 50° C. for at least 1 h, cooled down to 10° C. over at least 2 h, heat back up to 50° C. over at least 30 minutes, held at 50° C. for at least 2 h and finally cooled to 10° C. over at least 5 h. After holding the mixture at 10° C. for at least 5 h, water (3.04 L/Kg, 7 L) was charged to the reactor and the mixture was heated at 75° C. for at least 45 minutes, and held at that temperature for at least 15 minutes. The mixture was then cooled to 65° C. over at least 15 minutes, held at 65° C. for at least 1 h and cooled to 20° C. over at least 2 h. Then, 2-propanol (12.17 L/Kg, 28 L) was charged over at least 30 minutes via a head tank and stirring was maintained for at least 1 h., then the mixture was heated at 50° C. for at least 2 h, held at 50° C. for at least 1 h, and cooled to 10° C. over at least 5 h. After stirring at 10° C. for at least 5 h, the slurry was filtered on a Nutsche (Trade Mark) filter, and the cake was washed with cold 2-propanol/water (95:5 v/v, 2.5 L). The cake wash was used to rinse the reactor. The solids were placed on three oven trays. The trays were placed in a sealed vacuum oven at ambient temperature for 17 hours (along with a covered tray of water at the bottom of the oven to allow for humidifies drying and full rehydration) to provide the title compound (2.60 Kg, 4.61 mol, 90%, off-white solid).

### Example 4

### PXRD Analysis of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

A sample of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate prepared by the method of Example 3 was analysed by PXRD and the data was collected on a Bruker-AXS Ltd. D4 powder X-ray diffractometer (Trade Mark) fitted with an automatic sample changer, a theta-2 theta goniometer, motorised beam divergence slit, and a PSD Vantec-1 detector. The X-ray tube voltage and amperage were set to 35 kV and 40 mA respectively. Data was collected at the Cu K-alpha (wavelength 1.54 Å) using a step size of 0.018 degrees and scan time of 11.3 hours scanning from 2.0 to 65.0 degrees 2-theta. The sample was prepared by placing the powder in Si low background cavity holder. The sample powder was pressed by a glass slide to ensure that a proper sample height was achieved. Data were collected using Bruker DIFFRAC software (Trade Mark) and analysis was performed by DIFFRAC EVA software (Version 3.1) (Trade Mark). The PXRD patterns collected were imported into Bruker DIFFRAC EVA software (Trade Mark). The peak selection carried out manually was checked to ensure that all peaks below 25 degrees 2-theta had been captured and that all peak positions had been accurately assigned. A typical error of ±0.2° 2-theta in peak positions applies to these data. The ±0.2° 2-theta error associated with this measurement can occur as a result of a variety of factors including: (a) sample preparation (e.g., sample height), (b) instrument, (c) calibration, (d) operator (including those errors present when determining the peak locations), and (e) the nature of the material (e.g. preferred orientation and transparency errors). Therefore, peaks are considered to have a typical associated error of ±0.2° 2-theta. When two peaks, in the list, are considered to overlap the less intense peak has been removed from the listing. Peaks existing as shoulders, on a higher intensity adjacent peak, have also been removed from the peak list. While the shoulders may be >0.2° 2-theta from the position of the adjacent peak, they are not considered as discernible from the adjacent peak. In order to obtain the absolute peak positions, the powder pattern should be aligned against a reference. This could be either the calculated PXRD pattern from the single crystal structure determination on the same compound solved at room temperature, or an internal standard. e.g. silica or corundum.

The PXRD pattern for crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate of Example 3 is provided in FIG. 1. The characterising peak list is provided in Table 1. Certain peaks are selected as characteristic peaks for the title compound of Example 1. It is to be noted that for the title compound of Example 1, two of the characteristic peaks listed in Table 1 occur at 10.5 and 10.7° 2-theta. Whilst the separation of these peak positions lie at the limit of the 0.2° 2-theta acceptable error in peak position as described above, these peaks are to be considered as discrete peaks.

TABLE 1

| Angle °2-theta | % Relative Intensity |
|---|---|
| 4.5 | 5.0 |
| 5.8* | 100.0 |
| 7.2 | 11.2 |
| 8.9* | 18.1 |
| 10.5* | 69.5 |
| 10.7* | 71.3 |
| 11.5* | 58.2 |
| 12.2 | 30.4 |
| 13.1 | 2.3 |
| 14.7 | 65.3 |
| 15.3 | 7.3 |
| 16.6 | 13.9 |
| 17.5* | 71.4 |
| 18.0 | 66.2 |
| 18.6 | 16.2 |
| 19.3 | 14.2 |
| 21.0 | 26.7 |
| 21.6 | 13.4 |
| 22.7 | 14.6 |
| 23.1 | 61.1 |
| 23.5 | 86.0 |
| 24.8 | 26.3 |

(asterisked peak positions represent characteristic peaks)

Comparison of the data in Table 1 with that presented in Table 1A (see Example 5) for the calculated PXRD pattern of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate obtained from single crystal structure determination, shows a good characterising peak correlation indicative of the fact that the sample is crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate. The lack or loss of resolution of some peaks in FIG. 1 (compared to Table 1A) is to be expected and may be due to the inherent imperfection of the experimental data associated with (a) sample preparation (e.g., sample height and mass), (b) instrument, (c) calibration, (d) operator, and/or (e) the nature of the material (e.g. preferred orientation).

### Example 5

### Calculated PXRD Pattern of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

The calculated PXRD pattern of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate was obtained from single crystal structure determination. The single crystal was grown in 2-propanol/water and crystal structure was solved from this crystal as described in Example 9. The calculated PXRD powder pattern was obtained by a calculation using Reflex/Powder Diffraction Toolbox in Materials Studio 2018 software package (Trade Mark) from the solved crystal structure. The single crystal structure determination for crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate is shown in FIG. 5. The peak listing for the calculated PXRD pattern of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate obtained from single crystal structure determination is shown in Table 1A. The calculated PXRD pattern of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate contains all possible peaks that could possibly be observed in a PXRD pattern for this polymorph. It is to be expected that that not all possible peaks will be detected in an experimentally determined PXRD pattern. This may be due to the inherent imperfection of the experimental data associated with (a) sample preparation (e.g., sample height and mass), (b) instrument, (c) calibration, (d) operator, and (e) the nature of the material e.g. preferred orientation). Therefor the peak table for the calculated PXRD generally has more peaks than the experimental PXRD pattern.

TABLE 1A

| Angle °2-theta | % Relative Intensity |
|---|---|
| 4.5 | 14.7 |
| 5.8 | 100.0 |
| 7.2 | 6.5 |
| 8.9 | 16.6 |
| 10.5 | 32.7 |
| 10.7 | 46.7 |
| 11.6 | 17.7 |
| 11.8 | 3.1 |
| 12.3 | 13.1 |
| 12.5 | 2.6 |
| 13.2 | 7.0 |
| 14.1 | 6.1 |
| 14.4 | 5.0 |
| 14.7 | 21.2 |
| 15.3 | 23.8 |
| 16.4 | 7.0 |
| 16.6 | 29.4 |
| 17.3 | 2.3 |
| 17.5 | 23.0 |
| 17.8 | 6.0 |
| 18.0 | 16.8 |
| 18.1 | 11.0 |
| 18.3 | 5.6 |
| 18.6 | 6.2 |
| 19.3 | 3.7 |
| 21.0 | 28.0 |
| 21.1 | 6.7 |
| 21.6 | 13.5 |
| 21.9 | 45.1 |
| 22.7 | 44.4 |
| 23.1 | 49.8 |
| 23.5 | 14.7 |
| 23.7 | 19.5 |
| 24.2 | 5.0 |
| 24.3 | 5.5 |
| 24.5 | 3.7 |
| 24.8 | 31.3 |

### Example 6

### Solid State ¹³C-NMR (¹³C-ssNMR) of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

A sample of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate prepared by the method of Example 3 was analysed by ¹³C-ssNMR. The ¹³C-ssNMR spectrum is provided in FIG. 2. The peak list is provided in Table 2. The ¹³C-ssNMR analysis was conducted on a Bruker-BioSpin Avance III HD 400 MHz (1H frequency) NMR spectrometer (Trade Mark). The data were collected on a 4 mm MAS probe at a magic angle spinning rate of 10 kHz. The temperature was regulated to 20° C. Cross-polarization (CP) spectra with TOSS spinning sideband suppression were recorded with a 1 ms CP contact time and recycle delay of 3 seconds. A phase modulated proton decoupling field of ˜70 kHz was applied during spectral acquisition. The number of scans was adjusted to obtain an adequate signal to noise ratio and 3600 scans were collected. The ¹³C chemical shift scale was referenced using an external standard of crystalline adamantane, setting its down-field resonance to 38.5 ppm.

Automatic peak picking was performed using Bruker-BioSpin TopSpin version 3.2 software (Trade Mark). Generally, a threshold value of 3% relative intensity was used to preliminary select peaks. The output of the automated peak picking was visually checked to ensure validity and adjustments were manually made if necessary. Although specific ¹³C-ssNMR peak values are reported herein there does exist a range for these peak values due to differences in instruments, samples, and sample preparation. A typical variability for a ¹³C chemical shift x-axis value is of the order of plus or minus 0.2 ppm for a crystalline solid. The ¹³C-ssNMR peak heights reported herein are relative intensities. The ¹³C-ssNMR intensities can vary depending on the actual setup of the experimental parameters and the thermal history of the sample.

TABLE 2

| ¹³C Chemical Shifts [ppm] | % Relative Intensity |
|---|---|
| 16.2 | 69 |
| 17.9 | 26 |
| 19.9 | 88 |
| 29.7 | 16 |
| 32.4 | 27 |
| 37.9 | 17 |
| 40.1* | 73 |
| 54.5 | 32 |
| 58.9 | 38 |
| 70.6 | 90 |
| 77.0 | 35 |
| 78.7 | 46 |
| 79.8 | 40 |
| 83.1 | 42 |
| 99.9 | 32 |
| 105 | 25 |
| 108.7 | 33 |
| 110.3 | 34 |
| 116.5 | 68 |
| 117.5 | 69 |
| 121.3* | 94 |
| 123.5* | 109 |
| 124.5 | 65 |
| 126.9 | 20 |
| 145.1 | 26 |
| 148.5 | 26 |
| 149.3* | 96 |
| 150.7 | 59 |
| 151.3* | 100 |
| 154.0 | 57 |
| 158.2 | 55 |

(asterisked peak positions represent characteristic peaks)

### Example 7

### Solid State ¹⁹F-NMR (¹⁹F-ssNMR) of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

A sample of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate prepared by the method of Example 3 was analysed by ¹⁹F-ssNMR. The ¹⁹F-ssNMR spectrum is provided in FIG. 3. The peak list is provided in Table 3. The ¹⁹F-ssNMR analysis was conducted using the same spectrometer as used for the ¹³C-ssNMR analysis above. The data were collected on a 3.2 mm MAS probe at a magic angle spinning rate of 20 kHz. The temperature was regulated to 20° C. Cross-polarization (CP) spectra were recorded with a 400 μs CP contact time and recycle delay of 3 seconds. A phase modulated proton decoupling field of ˜65 kHz was applied during spectral acquisition. The number of scans was adjusted to obtain an adequate signal to noise ratio and 256 scans were collected. The ¹⁹F chemical shift scale was referenced using an external standard of trifluoroacetic acid and water (50/50 volume/volume), setting its resonance to −76.54 ppm (relative to CFCl₃). Automatic peak picking was performed using Bruker-BioSpin TopSpin version 3.2 software (Trade Mark). Generally, a threshold value of 3% relative intensity was used to preliminary select peaks. The output of the automated peak picking was visually checked to ensure validity and adjustments were manually made if necessary. Although specific ¹⁹F-ssNMR peak values are reported herein there does exist a range for these peak values due to differences in instruments, samples, and sample preparation. A typical variability for a ¹⁹F chemical shift x-axis value is of the order of plus or minus 0.2 ppm for a crystalline solid. The ¹⁹F-ssNMR peak heights reported herein are relative intensities. The ¹⁹F-ssNMR intensities can vary depending on the actual setup of the experimental parameters and the thermal history of the sample.

TABLE 3

| ¹⁹F Chemical Shifts [ppm] | % Relative Intensity |
|---|---|
| −129.6* | 100 |
| −128.4* | 97 |
| −109.8 | 52 |
| −108.8 | 51 |
| −107.8 | 50 |
| −106.0 | 43 |

(asterisked peak positions represent characteristic peaks)

### Example 8

### FT Raman Spectroscopy of Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

A sample of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate prepared by the method of Example 3 was analysed by FT Raman spectroscopy. The FT Raman spectrum is provided in FIG. 4. The peak list is provided in Table 4. Raman spectra were collected using a RAM II FT Raman module attached to a Bruker Vertex 70 FTIR spectrometer (Trade Mark). The instrument is equipped with a 1064 nm Nd:YAG laser and a liquid nitrogen cooled germanium detector. Prior to data acquisition, instrument performance and calibration verifications were conducted using a white light source, and polystyrene and naphthalene references. Samples were prepared and analysed in truncated NMR tubes (5 mm diameter). A sample rotator (Ventacon) was used during measurement to maximise the volume of material exposed to the laser during data collection. The backscattered Raman signal from the sample was optimised and data were collected at a spectral resolution of 2 cm⁻¹, using a laser power of 500 mW. A Blackmann-Harris 4-term apodization function was applied to minimise spectral aberrations. Spectra were generated between 3500 and 200 cm⁻¹ with the number of scans adjusted accordingly to ensure adequate signal to noise. Spectra were normalised by setting the intensity of the most intense peak to 1.00. Peaks were then identified using the automatic peak picking function in the GRAMS/AI v9.2 software (Thermo Fisher Scientific) with the threshold set to 0.05. Peak positions and relative peak intensities were extracted and tabulated, with peaks then being categorised as very strong (vs), strong (s), medium (m), and weak (w) for the intensity ranges 1.00-0.75, 0.74-0.50, 0.49-0.25, and <0.25 respectively. The variability in the peak positions with this experimental configuration is within ±2 cm⁻¹. It is expected that, since FT Raman and dispersive Raman are similar techniques, peak positions reported in this document for FT Raman spectra would be consistent with those which would be observed using a dispersive Raman measurement, assuming appropriate instrument calibration.

TABLE 4

| Peak position cm⁻¹ (±2 cm⁻¹) | Relative intensity |
|---|---|
| 233 | m |
| 316 | m |
| 349 | m |
| 430 | m |
| 460 | m |
| 492 | m |
| 564 | m |
| 603 | m |
| 669 | m |
| 702* | s |
| 718 | m |
| 827 | m |
| 850 | m |
| 907 | m |
| 960 | m |
| 1046 | m |
| 1158 | m |
| 1239 | m |
| 1266 | m |
| 1325 | m |
| 1342 | m |
| 1369 | m |
| 1392 | m |
| 1438 | m |
| 1471 | m |
| 1512 | vs |
| 1604* | m |
| 1630* | m |
| 2924 | s |
| 2972 | m |
| 3006 | m |
| 3023 | m |

(asterisked peak positions represent characteristic peaks and relative peak intensity is denoted as being very strong (vs), strong (s), medium (m), or weak (w))

### Example 9

### Single Crystal Structure Determination for Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

Data collection was performed on a Bruker D8 Venture diffractometer at room temperature. Data collection consisted of omega and phi scans. The structure was solved by intrinsic phasing using SHELX software suite in the Monoclinic space group P21 with the following cell parameters: a=15.3572(6) Å; b=8.1080(3) Å; c=19.9014(8) Å; alpha=90°; beta=91.447(2°); gamma=90°. The structure was subsequently refined by the full-matrix least squares method. All non-hydrogen atoms were found and refined using anisotropic displacement parameters. The hydrogen atoms located on nitrogen and oxygen were found from the Fourier difference map and refined with distances restrained. The remaining hydrogen atoms were placed in calculated positions and were allowed to ride on their carrier atoms. The final refinement included isotropic displacement parameters for all hydrogen atoms. As noted in the figure, one of the water molecules is given without hydrogen atoms bonded. Lattice contains two waters molecules in the asymmetric unit: one water with full occupancy and one water position with approximately 0.2 occupancy. Overall, the ratio of API to water is approximately 1 to 1.1. Analysis of the absolute structure using likelihood methods (Hooft 2008) was performed using PLATON (Spek 2010). Assuming the sample submitted is enantiopure, the results indicate that the absolute structure has been correctly assigned. The final R-index was 3.9%. A final difference Fourier revealed no missing or misplaced electron density. FIG. 5 depicts asymmetric unit of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate with displacement parameters drawn at 50% probability. The hydrogen on water molecule O3W is omitted.

### Reference Example 1

### Repeat of the Preparation of the Compound (1S,2S,3S,5R)-3-((6-(difluoromethyl)-5-fluoro-1,2,3,4-tetrahydroisoquinolin-8-yl)ox)-5-(4-methyl-7H-pyrrolo[2,3-d]pyrimidin-7-yl)cyclopentane-1,2-diol as a hydrochloride salt (ZZZ-16), as Described in Example 190 of WO2017/212385

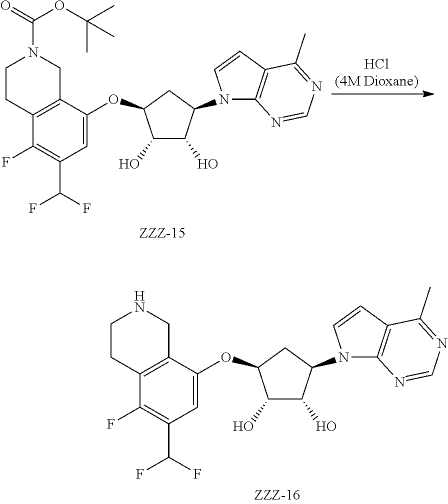 [SMILES: Cc1ncnc2c1ccn2[C@@H]1C[C@H](Oc2cc(C(F)F)c(F)c3c2CN(C(=O)OC(C)(C)C)CC3)[C@@H](O)[C@H]1O ; Cc1ncnc2c1ccn2[C@@H]1C[C@H](Oc2cc(C(F)F)c(F)c3c2CNCC3)[C@@H](O)[C@H]1O]

To a solution of ZZZ-15 (210 mg, 0.383 mmol) in dichloromethane (2 mL) was added a 4M solution of HCl in dioxane (0.766 mL, 3.06 mmol) at 0° C. in a 25 mL pear-shaped round bottom flask. The ice bath was removed and the mixture was stirred at room temp (23° C.) for 2 hours. A white solid precipitated, sticking to the slides of the flask. LCMS indicates >95% conversion. The clear liquid was removed by pipette and the solid dried under reduced pressure. The solid was dissolved in 4 mL water and freeze dried by lyophilization for 70 hours to give ZZZ-16 as an amorphous off white solid (184 mg, 92%). LCMS [M+1]=449; ¹H NMR (400 MHz, D₂O): δ=8.85 (s, 1H), 7.82 (br. s., 1H), 7.18-6.87 (m, 3H), 5.41-5.31 (m, 1H), 4.86-4.81 (m, 1H), 4.72-4.69 (m, 1H), 4.40 (br. s., 2H), 4.36-4.33 (m, 1H), 3.53 (t, J=6.0 Hz, 2H), 3.16-3.03 (m, 3H), 2.91 (s, 3H), 2.27-2.15 (m, 1H) ppm. [Equivalent to ZZZ-16 produced in Example 190 of WO2017/212385: LCMS [M+1] 449; 1H NMR (400 MHz, D2O) δ ppm 8.93 (s, 1H), 7.91 (d, J=3.8 Hz, 1H), 7.24-6.88 (m, 3H), 5.41 (q, J=9.0 Hz, 1H), 4.87 (br dd, J=2.4, 4.4 Hz, 1H), 4.75 (dd, J=5.0, 8.8 Hz, 1H), 4.45 (s, 2H), 4.39 (br d, J=5.0 Hz, 1H), 3.58 (t, J=6.3 Hz, 2H), 3.19-3.07 (m, 3H), 2.99 (s, 3H), 2.35-2.24 (m, 1H).]

### Reference Example 2

### Elemental Analysis of the (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol hydrochloride salt of Reference Example 1

The product of Reference Example 1 was shown to contain 2 moles HCl and ˜1 H2O per mole, based on elemental analysis, Elemental analysis was performed by Atlantic Microlab, Inc. (Norcross Ga.). Samples were weighed on electronic microbalances (Perkin-Elmer Model AD4 or Model AD6; Mettler Model MTS; Cahn Model 30, Model 31, Model 33 or Model 34) calibrated daily prior to the weighing of any samples. Carbon, hydrogen and nitrogen analyses were performed on automatic analyzers which utilize a technique based on a modification of the classical Pregl and Dumas methods. Analyzers were: Perkin-Elmer Model 2400 Series II Auto-analyzers or Carlo Erba Model 1108 Analyzers, calibrated daily with ultra-high purity standards prior to the analysis of any samples. Instrument specifications list a precision of +/−0.3 percent. Samples were weighed and then introduced into an auto-analyzer which is maintained under a positive pressure with the carrier gas of helium. Fluorine, Chlorine, Bromine, and Iodine were performed by Schoniger Flask Combustion followed by analysis using Ion Chromatography. The sample was diluted, filtered and injected into the IC. The data is processed to yield the PPM of each halogen, and then converted to percentages by the following calculation: PPM X Volume (L)/sample weight (KG)×10000 (10000 PPM=1%). The results of elemental analysis are presented in Table 6:

TABLE 6

| Element | Theory | Found (run 1) | Found (run 2) |
|---|---|---|---|
| C | 48.99 | 48.75 | 48.93 |
| H | 5.05 | 5.10 | 5.10 |
| N | 10.39 | 10.06 | 10.03 |
| Cl | 13.14 | 12.76 | 12.79 |

This elemental analysis shows the product to be a dihydrochloride 1.0-1.28 hydrate of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol.

### Reference Example 3

### PXRD Analysis of the (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol hydrochloride salt of Reference Example 1

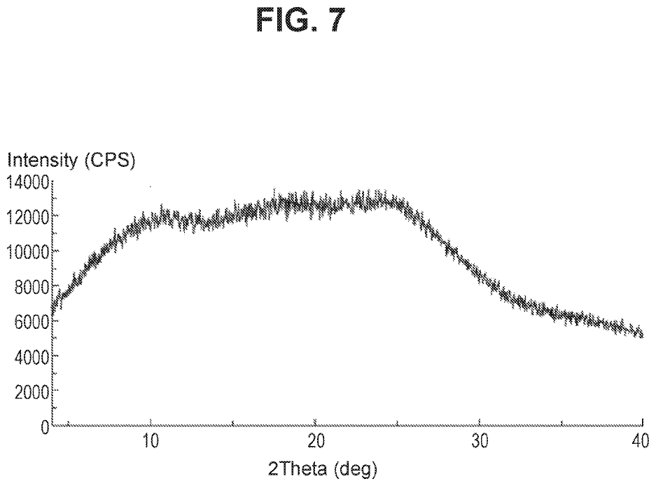

PXRD analysis of the reference Example 1 product show it to be amorphous. Analysis was performed using a Rigaku Miniflex 600 diffractometer. Data collected over a range of 4 to 40 degrees using Cu radiation at a power of 15 mA and 40 kV. The PXRD pattern indicates that an amorphous product was obtained. See FIG. 7.

### Comparative Example 1

### Hygroscopicity of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol as a hydrochloride salt compared to crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate

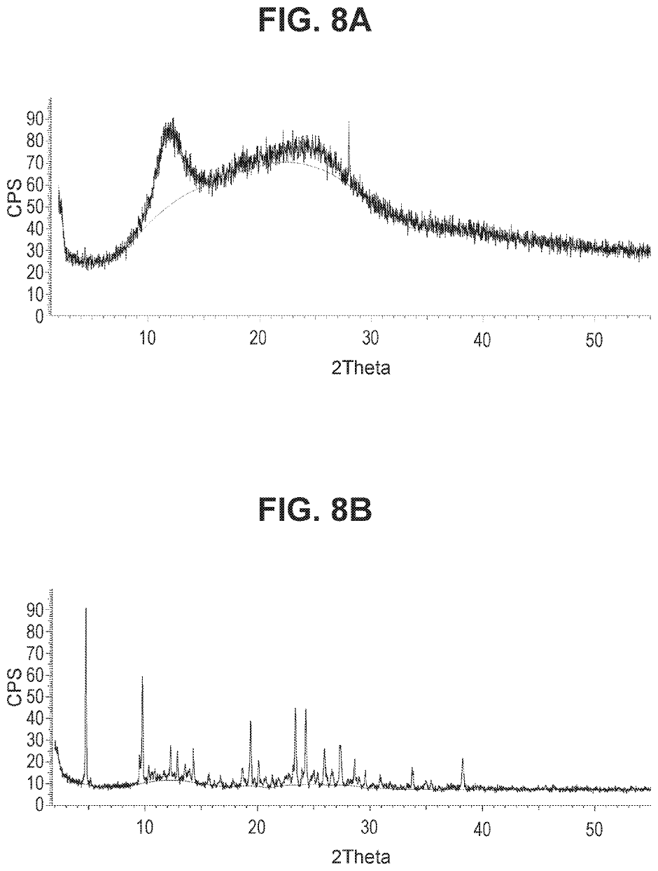

The hygroscopicity of the products of Reference Example 1 and Example 3 were tested using Dynamic Vapor Sorption (DVS). Surface Measurement Systems Ltd. Dynamic Vapour Sorption Advantage Equipment was used employing a 10% RH change at 25° C. starting at 40% RH and looping from 0% RH to 90% RH and back to 0% RH twice. The Reference Example 1 di HCl salt material was gradually exposed to increasing relative humidity and the sample mass was recorded. Two full cycles from 0% RH to 90% RH were carried out. The percent mass increase with respect to the sample mass of the first exposure to 0% RH (dry weight) was calculated and is presented in Table 7. The crystallinity of the Reference Example 1 material was checked by PXRD after the DVS run and showed that material had converted to a crystalline solid. DVS data for the Example 3 phosphate hydrate was similarly generated and is provided in Table 7 as well. The DVS data suggest that the diHCl salt is very hygroscopic, increasing its mass by 14.7% at 70% RH. Also, a decrease in mass is noted at 80% RH consistent with a solid-state change, occurs at these conditions. This is supported by the PXRD analysis (FIG. 8) after the DVS run, confirming that the di-HCL salt is not physically stable, is very hygroscopic, and crystallizes when exposed to RH≥80% RH. In contrast to the diHCl salt, the Example 3 phosphate salt increases its mass only by 4.57% at 70% RH (mono-hydrate stoichiometry relates to 3.3%). A hydration (1 mole water equivalent) occurs at 10% RH and mass increases gradually goes up from 10% RH to 90% RH consistent with there being no structural changes caused by the water sorption.

TABLE 7

| RH | di HCl (% Mass-Ref Dry mass) | Phosphate % Mass-Ref Dry mass |
|---|---|---|
| 0 | 0.0 | 0.0 |
| 10 | 2.9 | 3.4 |
| 20 | 4.0 | 3.7 |
| 30 | 4.9 | 3.9 |
| 40 | 6.0 | 4.1 |
| 50 | 7.6 | 4.3 |
| 60 | 10.4 | 4.5 |
| 70 | 14.7 | 4.6 |
| 80 | 12.0 | 4.7 |
| 90 | 12.0 | 4.8 |

Modifications may be made to the foregoing without departing from the basic aspects of the invention. Although the invention has been described in substantial detail with reference to one or more specific embodiments, those of ordinary skill in the art will recognise that changes may be made to the embodiments specifically disclosed in this application, and yet these modifications and improvements are within the scope and spirit of the invention.

1. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate.

2. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 characterised by a PXRD pattern measured using Cu K-alpha radiation comprising at least three characterising peaks selected from about 5.8, 10.5, 10.7, 11.5 and 17.5 degrees 2-theta (+/−0.2 degrees 2-theta).

3. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 2 characterised by a PXRD pattern measured using Cu K-alpha radiation comprising characterising peaks at about 5.8, 10.5 and 10.7 degrees 2-theta (+/−0.2 degrees 2-theta).

4. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 2 characterised by a PXRD pattern measured using Cu K-alpha radiation comprising characterising peaks at about 5.8, 11.5 and 17.5 degrees 2-theta (+/−0.2 degrees 2-theta).

5. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 3 characterised by a PXRD pattern measured using Cu K-alpha radiation comprising characterising peaks at about 5.8, 10.5, 10.7 and 17.5 degrees 2-theta (+/−0.2 degrees 2-theta).

6. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 2 characterised by a PXRD pattern measured using Cu K-alpha radiation comprising characterising peaks at about 5.8, 10.5, 10.7, 11.5 and 17.5 degrees 2-theta (+/−0.2 degrees 2-theta).

7. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 6 characterised by a PXRD pattern measured using Cu K-alpha radiation comprising characterising peaks at about 5.8, 8.9, 10.5, 10.7, 11.5 and 17.5 degrees 2-theta (+/−0.2 degrees 2-theta).

8. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 2 characterised by a PXRD pattern measured using Cu K-alpha radiation essentially the same as shown in FIG. 1.

9. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 2 characterised by a PXRD pattern measured using Cu K-alpha radiation having a PXRD peak listing essentially the same as in Table 1.

10. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 characterised by a ¹³C-ssNMR spectrum comprising characterising peaks at about 123.5 and 149.3 ppm±0.2 ppm.

11. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 10 characterised by a ¹³C-ssNMR spectrum comprising characterising peaks at about 40.1, 123.5 and 149.3 ppm±0.2 ppm, at about 40.1, 121.3, 123.5 and 149.3 ppm±0.2 ppm, or at about 40.1, 121.3, 123.5, 149.3 and 151.3 ppm±0.2 ppm.

12. (canceled)

13. (canceled)

14. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 10 characterised by a ¹³C-ssNMR spectrum essentially the same as shown in FIG. 2.

15. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 10 characterised by a ¹³C-ssNMR spectrum peak listing essentially the same as in Table 2.

16. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 characterised by a ¹⁹F-ssNMR spectrum comprising a characterising peak at about −129.6 ppm±0.2 ppm or at about −129.6 and −128.4 ppm±0.2 ppm.

17. (canceled)

18. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 16 characterised by a ¹⁹F-ssNMR spectrum essentially the same as shown in FIG. 3.

19. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 16 characterised by a ¹⁹F-ssNMR spectrum peak listing essentially the same as in Table 3.

20. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 characterised by a FT Raman spectrum comprising characterising peaks at about 702 and 1630 cm⁻¹±2 cm⁻¹ or at about 702, 1604 and 1630 cm⁻¹±2 cm⁻¹.

21. (canceled)

22. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 20 characterised by a FT Raman spectrum essentially the same as shown in FIG. 4.

23. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 20 characterised by a FT Raman spectrum peak listing essentially the same as in Table 4.

24. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 in substantially pure form.

25. (canceled)

26. Crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 wherein about 1.0 to about 1.4 molar equivalents of water per mole of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate are present.

27. A pharmaceutical composition comprising crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1 and a pharmaceutically acceptable carrier or excipient.

28. (canceled)

29. (canceled)

30. (canceled)

31. A method of treatment of abnormal cell growth in a mammal comprising administering to the mammal a therapeutically effective amount of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1, or a composition thereof as claimed in claim 27, wherein the abnormal cell growth is cancer.

32. (canceled)

33. A combination of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol monophosphate hydrate as claimed in claim 1, or a composition thereof as claimed in claim 27, with another anti-cancer agent.
